(19) United States
(12) Patent Application Publication
(10) Pub. No.: US 2013/0089583 A1
(43) Pub. Date: Apr. 11, 2013

(54) HAIR COLORATION COMPOSITIONS EXHIBITING PREDETERMINED STRUCTURAL COLORATION THROUGH PHOTONIC BAND GAP EFFECTS

(71) Applicant: Timothy J. Taylor, Phoenix, AZ (US)

(72) Inventor: Timothy J. Taylor, Phoenix, AZ (US)

(73) Assignee: The Dial Corporation, Scottsdale, AZ (US)

(21) Appl. No.: 13/267,482

(22) Filed: Oct. 6, 2011

(51) Int. Cl. A61K 8/11; A61Q 5/06; A61K 8/81; A61Q 5/10; B82Y 99/00

(52) U.S. Cl. 424/401; 424/70.6; 8/405; 977/773

(57) ABSTRACT

Hair coloration compositions for application to hair are provided. In an embodiment, a hair coloration composition includes a plurality of particles. Each particle comprises a first layer of first material and a first layer of second material with an interface therebetween. The first material has a first refractive index and the second material has a second refractive index. The difference between the first refractive index and the second refractive index is greater than zero. Each particle is configured to exhibit a photonic band gap effect at the interface when exposed to incident white light.

### TECHNICAL FIELD

This document generally relates to hair coloration compositions, and more particularly relates to hair coloration compositions that exhibit photonic band gaps when exposed to incident white light to provide hair with a predetermined structural coloration.

### BACKGROUND

While attempts to alter the color of human hair have long been made, the attainment of natural colors that are retained by the hair for a desirable period has remained an elusive goal. Various approaches to hair dyeing have been developed, including direct action dyes, natural dyes, metallic dyes, oxidative dyes, pigments or other colorants.

Conventionally, coloring may be carried out by mixing these coloring materials into a cosmetic ingredient composition that is applied to the hair. Upon application, the coloring materials affix to the hair and/or dye the hair. In the case of such conventional coloring materials, differences may occur in the wavelength characteristics of the light reflected from the surface of the coloring material due to differences in the absorption spectrum characteristics at the surface of the coloring material. As a result, an observer perceives a coloration of the hair.

However, the coloring differences exhibited generally do not match the natural coloring of hair. Specifically, conventional coloring materials typically result in a monochromatic (single wavelength) color. Consumers do not desire a monochromatic color, as it appears unnatural. Rather, consumers seek colored hair that exhibits a diffuse spectrum of color, known as “highlights” together with a central color.

Further, typical pigments, colorants and dyes include irritants or even toxic components that are not entirely harmless with respect to the human body. Often these pigment, colorant, or dye molecules are small enough to pass through the skin's lipid boundary and be absorbed into the scalp, causing irritation and damage.

Accordingly, it is desirable to provide a hair coloration composition that exhibits photonic band gap effects when exposed to incident white light to more accurately mimic natural hair color. It is also desirable to provide a hair coloration composition that exhibits a predetermined structural coloration when exposed to incident white light. Also, it is desirable to provide a hair coloration composition that exhibits reduced skin absorption and irritation. Furthermore, other desirable features and characteristics of the hair coloration compositions will become apparent from the subsequent detailed description and the appended claims, taken in conjunction with the accompanying drawings and this background.

### BRIEF SUMMARY

Hair coloration compositions for exhibiting predetermined structural coloration through photonic band gap effects are provided. In accordance with one exemplary embodiment, a hair coloration composition comprises a plurality of particles. Each particle includes a first layer of first material and a first layer of second material defining an interface with the first layer of first material. The first material has a first refractive index and the second material has a second refractive index, such that the difference between the first refractive index and the second refractive index is greater than zero. Further, each of the plurality of particles is configured to exhibit a photonic band gap effect at the interface when exposed to incident white light.

In another embodiment, a hair coloration composition includes a plurality of particles, and each particle includes alternating first and second layers that define interfaces therebetween. Each layer is respectively formed from a first material and a second material. Further, the first material has a first refractive index and the second material has a second refractive index, such that the difference between the first refractive index and the second refractive index is greater than zero. Each particle is configured to exhibit a photonic band gap effect at the interfaces when exposed to incident white light. The hair coloration composition further includes a medium for holding the plurality of particles.

In accordance with another exemplary embodiment, a hair coloration composition is provided for application to keratinous fibers. The hair coloration composition includes at least one particle in a medium including alternating first and second layers that define interfaces therebetween. The first and second layers are respectively formed from a first material and a second material. Further, the first material has a first refractive index and the second material has a second refractive index, such that the difference between the first refractive index and the second refractive index is greater than zero. Also, the particle is configured to exhibit a photonic band gap effect at the interfaces when exposed to incident white light to provide a predetermined structural coloration.

### BRIEF DESCRIPTION OF THE DRAWINGS

Exemplary embodiments will hereinafter be described in conjunction with the following drawing figures, wherein like numerals denote like elements, and wherein:

FIG. 1 is a schematic view of the layers of a particle for use in a hair coloration composition in accordance with an exemplary embodiment;

FIG. 2 is a cross-sectional view of a particle from a hair coloration composition in accordance with an exemplary embodiment; and

FIG. 3 is a schematic view of a hair coloration composition for exhibiting a predetermined structural coloration in accordance with an exemplary embodiment.

### DETAILED DESCRIPTION

The following detailed description is merely exemplary in nature and is not intended to limit the hair coloration compositions or the application and uses of the hair coloration compositions. Furthermore, there is no intention to be bound by any theory presented in the preceding background or the following detailed description.

The hair coloration compositions contemplated herein utilize a coloration principle distinct from conventional coloring methods that rely on the absorption and reflection of differing portions of light through pigmentation. Instead, the present hair coloration compositions provide a structural coloration to hair. Specifically, the present hair coloration compositions exhibit a selected photonic band gap effect when exposed to incident white light to provide a desired color reflection. As used herein, “photonic band gap” refers to a wavelength range for which a material neither absorbs light nor allows light propagation. Band gaps are known from photonic crystals that have a periodic pattern of materials of different refractive indices. When such a crystal has a periodic pattern that is equal to half the length of a given monochromatic light wavelength, it does not allow propagation of that wavelength. When light is irradiated onto the crystal, the wavelengths within the band gap are reflected.

The hair coloration compositions herein employ the band gap effect to perform a desired color reflection by selectively reflecting only predetermined wavelengths of light. For example, if red is the desired color to be reflected, then the hair coloration composition is designed to reflect a plurality of wavelengths centered at 800 nm (nanometers) while other wavelengths are destructed or allowed to pass without reflection. As a result, application of the hair coloration composition to hair provides a diffuse red coloration to the hair.

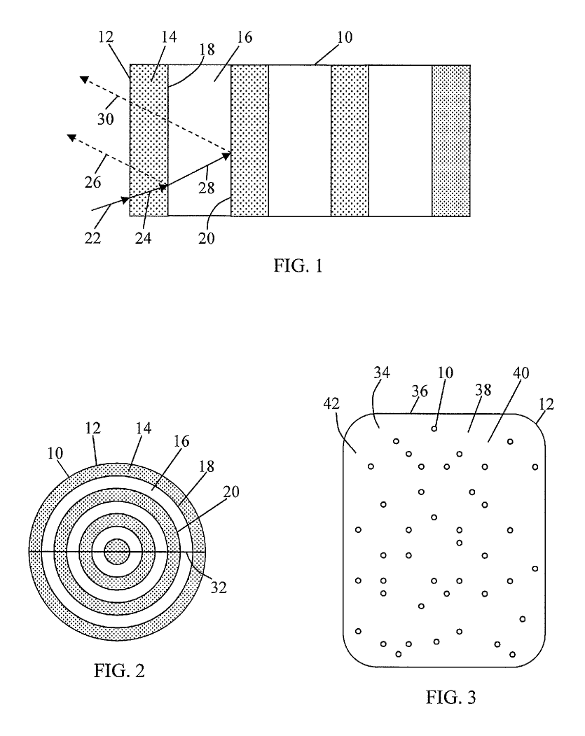

FIG. 1 illustrates the coloration effect of a simplified two layer particle 10 for use in the hair coloration compositions herein. The hair coloration compositions are useful for the coloration of human and animal keratinous fibers, including hair, eyebrows, eyelashes, and fur. As shown, the particle 10 includes a surface 12, a first layer 14 of a first material, a second layer 16 of a second material, a first interface 18, and a second interface 20. It is understood, that the particle may include a large number of layers 14 and 16. For instance, there may be hundreds of layers 14 and 16. Further, in alternate embodiments, the particle 10 may include layers of third materials, layers of fourth materials, and so on, alternating with the first and second layers 14 and 16. While there is no defined upper limit to the number of layers, the particle 10 must have at least one first layer 14 and one second layer 16.

FIG. 1 illustrates a path of light 22 incident on the surface 12 of the particle 10. As is well understood, light is reflected whenever it travels from a medium of a given refractive index into a medium with a different refractive index. When passing through media with very similar refractive indices, little light may be reflected. Instead, most of the light is refracted in the second medium.

As the particle 10 herein is intended for use with light passing through the air, the first material is selected to have a refractive index (n_{A}) very similar to air's refractive index, i.e., close to 1.0, preferably between about 0.95 and about 1.05, and most preferably between about 0.9 and about 1.1. As a result, very little light is reflected at the surface 12 of the particle 10 when light passes from air to the particle 14. Instead, light 22 incident on the surface 12 of the particle 10 moves as refracted light 24 through the first layer 14.

The second material of particle 10 is selected to provide a refractive index (n_{B}) distinct from the first material's refractive index to maximize reflection at the first interlayer interface 18. To provide sufficient reflection, the index of refraction difference Δn, equal to the absolute value of n_{A}−n_{B}, is preferably greater than about 0.05, more preferably greater than about 0.1, and most preferably greater than about 0.5.

As shown, reflected light 26 is reflected at the first interlayer interface 18. Some refracted light 28 may pass further into particle 10 through second layer 16. As the refractive index difference (Δn) at the second interlayer interface 20 is equal in magnitude to the difference (Δn) at the first interlayer interface 18, a large portion of the light 28 reflects as reflected light 30. As may be imagined, a decreasing amount of light may continue to propagate through the alternating layers 14 and 16, while portions of the light are reflected at each interface 18 and 20.

To provide a reflection of selected wavelengths and the desired coloration effect, the particle 10 is designed to exhibit a photonic band gap. The photonic band gap is related to the thickness (d_{B}) of the second layer 16 via the Bragg diffraction equation:

λ=2d_{B}n_{B} sin θ

wherein λ is the wavelength diffracted, n_{B} is the refractive index of the second material, and θ is the Bragg diffraction angle. From the Bragg diffraction equation, the photonic band gap will be a maximum when sin θ=1 (i.e., when θ=90°), and θ=90° for incident light perpendicular to the first interlayer interface 18.

Therefore, the particle 10 can be designed with a selected first material, second material, and second layer thickness to provide a maximized reflection of a desired color. For instance, a desired coloration may be selected. Therefore, the corresponding wavelength (λ) is known. Further, second material may be selected with a known refractive index (n_{B}) to maximize reflection at the first interlayer interface 18. With these variables selected, the required thickness (d_{B}) of the second layer 16 is calculated from the reorganized equation:

d_{B}=λ/2N_{B}.

In FIG. 2, the reflected light 26 will be comprised of the wavelengths within the photonic band gap because those wavelengths cannot pass through the second layer 16. Wavelengths that are not within the photonic band gap will largely pass through the layers 14 and 16. As a result, the predetermined structural coloration is achieved.

FIG. 2 illustrates an exemplary structure of a particle 10. As shown, particle 10 includes alternating layers 14 and 16 that are respectively formed from the selected first layer material and the selected second layer material. Each layer material may be a polymer, an inorganic material, or a self-organized organic material. While FIG. 2 illustrates a substantially spheroidal particle 10, it is envisioned that the particles 10 may be rod-shaped, lamellar, or have other shapes as desired.

Particle 10 has a largest dimension 32, such as a diameter, a length, or the like, that is preferably between about 1 micron and about 1000 microns, more preferably between about 10 microns and about 500 microns, and most preferably between about 25 microns and about 250 microns. It is important that the largest dimension 32 of each particle 10 be at least about 1 micron so that particles 10 are not able to cross the skin's lipid boundary and are not absorbed into the user's skin. The thickness of each layer 14 and 16 is between about 1 nm and about 1000 nm, preferably between about 2.5 nm and about 500 nm, and most preferably between about 5 nm and about 300 nm. Further, the thickness of layers 14 may differ from the thickness of layers 16. While the exemplary particle in FIG. 2 includes seven layers for purposes of clarity, the particle 10 includes at least two layers, and may include hundreds of layers.

Referring now to FIG. 3, a hair coloration composition 34 is shown in a container 36. The hair coloration composition 34 may be in the form of a cream, gel, oil, or other form facilitating application to the hair. As shown, the hair coloration composition 34 includes a plurality of the particles 10. The particles 10 may be of differing sizes with differing largest dimensions 32. Further, the particles 10 may have different layers, as well as different numbers of layer materials in order to provide a diffuse spectrum of coloration.

As shown, the hair coloration composition 34 includes a medium 38 in which the particles 10 are carried. The medium 38 may be any of the known creams, gels, oils, or other fluids which are used commercially as carriers in hair care products, provided that it does not react with particles 10. For example, the medium may be a liquid having polarity, such as water, one or more C₁-C₁₂-monoalcohol, for example one or more C₂-C₆-monoalcohol, methanol, ethanol, or mixtures thereof, a hydrophilic medium comprising water or a mixture of water and hydrophilic organic solvents, for instance alcohols, such as linear or branched lower monoalcohols containing from 2 to 5 carbon atoms, for instance ethanol, isopropanol or n-propanol, and polyols, for instance glycerol, diglycerol, propylene glycol, sorbitol or pentylene glycol, and polyethylene glycols, or alternatively hydrophilic C₂ ethers and C₂-C₄ aldehydes. The water or the mixture of water and hydrophilic organic solvents may be present in the composition 34 according to an embodiment in an amount ranging from 0.1% to 99% by weight, for instance from 10% to 80% by weight, relative to the total weight of the composition 34.

The medium 38 may comprise fatty substances that are liquid at room temperature and/or of fatty substances that are solid at room temperature, such as waxes, pasty fatty substances and gums, and mixtures thereof. These fatty substances may be of animal, plant, mineral or synthetic origin. This fatty phase may also contain lipophilic organic solvents. As fatty substances that are liquid at room temperature, often referred to as oils, which may be used in the invention, non-limiting mention may be made of hydrocarbon-based oils of animal origin such as perhydrosqualene; hydrocarbon based plant oils such as liquid triglycerides of fatty acids of 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter; linear or branched hydrocarbons of mineral or synthetic origin, such as liquid paraffin and derivatives thereof, petroleum jelly, polydecenes, hydrogenated polyisobutene such as parleam; synthetic esters and ethers, especially of fatty acids, for instance purcellin oil, isopropyl myristate, 2-ethylhexyl palmitate, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol and oleyl alcohol; partially hydrocarbon-based fluoro oils and/or partially silicone-based fluoro oils; volatile or non-volatile silicone oils, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxydiphenylsiloxanes, diphenylmethyldimethyltrisiloxanes, diphenyl dimethicones, phenyl dimethicones and polymethylphenylsiloxanes; and mixtures thereof. These oils may be present in an amount ranging from 0.01% to 90%, for instance from 0.1% to 85% by weight relative to the total weight of the composition 34.

The medium 38 may also comprise one or more physiologically acceptable organic solvents. These solvents may be generally present in an amount ranging from 0.1% to 90%, for instance from 0.5% to 85%, for example from 10% to 80% and further still from 30% to 50% by weight, relative to the total weight of the composition 34. Non-limiting mention may be made for example, besides the hydrophilic organic solvents mentioned above, of ketones that are liquid at room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate, and dipropylene glycol mono-n-butyl ether; short-chain esters (containing from 3 to 8 carbon atoms in total), such as ethyl acetate, methyl acetate, propyl acetate, n-butyl acetate and isopentyl acetate; ethers that are liquid at room temperature, such as diethyl ether, dimethyl ether or dichlorodiethyl ether; alkanes that are liquid at room temperature, such as decane, heptane, dodecane, isododecane and cyclohexane; aromatic cyclic compounds that are liquid at room temperature, such as toluene and xylene; aldehydes that are liquid at room temperature, such as benzaldehyde and acetaldehyde, and mixtures thereof.

The composition 34 according to the present disclosure may also comprise at least one physiologically acceptable wax. For the purposes of the present disclosure, the term “wax” means a lipophilic compound that is solid at room temperature, which undergoes a reversible solid/liquid change of state, and which has a melting point of greater than or equal to 25° C., which may be up to 120° C. The waxes may be hydrocarbon-based waxes, fluoro waxes and/or silicone waxes and may be of plant, mineral, animal and/or synthetic origin. For instance, the waxes may have a melting point of greater than 30° C. and further still, for example, greater than 45° C. As disclosed herein, waxes that may be used in the composition 34 of the present disclosure, include but are not limited to those made of beeswax, carnauba wax or candellila wax, paraffin, microcrystalline waxes, ceresin or ozokerite, synthetic waxes, for instance polyethylene waxes or Fischer-Tropsch waxes, and silicone waxes, for instance alkyl or alkoxy dimethicones containing from 16 to 45 carbon atoms. The gums are generally polydimethylsiloxanes (PDMSs) of high molecular weight or cellulose gums or polysaccharides, and the pasty substances are generally hydrocarbon-based compounds, for instance lanolins and derivatives thereof, or PDMSs. The nature and amount of the solid substances depend on the desired mechanical properties and textures. As a guide, the composition 34 may contain from 0.1% to 50% by weight, for example, from 1% to 30% by weight of waxes relative to the total weight of the composition 34.

The composition 34 according to the present disclosure may also comprise, in a particulate phase, pigments and/or nacres and/or fillers usually used in cosmetic compositions. The composition 34 may also comprise other dyestuffs chosen from water-soluble dyes and/or liposoluble dyes that are well known to those skilled in the art. The term “pigments” should be understood as meaning white or colored, mineral or organic particles of any shape, which are insoluble in the physiological medium and which are intended to color the composition 34. The pigments may be present in the composition 34 in a proportion ranging from 0.01% to 25% and for example in a proportion of from 3% to 10% by weight of the final composition 34. They may be white or colored, and mineral or organic. Non-limiting mention may be made of titanium oxide, zirconium oxide or cerium oxide, and also zinc oxide, iron oxide or chromium oxide, ferric blue, chromium hydrate, carbon black, ultramarines (aluminosilicate polysulfides), manganese pyrophosphate and certain metallic powders such as silver or aluminium powder. Mention may also be made of calcium, barium, aluminium, strontium or zirconium salts.

The nacres may be present in the composition 34 in a proportion ranging from 0.01% to 20% by weight and further, for example, in a proportion ranging from 3% to 10% by weight. The term “nacres” should be understood as meaning iridescent particles of any form, produced especially by certain molluscs in their shell, or else synthesized. Among the nacres that may be envisioned, mention may be made of natural mother-of-pearl, mica coated with titanium oxide, with iron oxide, with natural pigment or with bismuth oxychloride, and also colored titanium mica. Non-limiting mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The composition 34 according to the present disclosure may also comprise one or more fillers, for instance in an amount ranging from 0.01% to 50% by weight and for example ranging from 0.02% to 30% by weight, relative to the total weight of the composition 34. The term “fillers” should be understood as meaning colorless or white, mineral or synthetic, lamellar or non-lamellar particles intended to give body or rigidity to the composition 34, and/or softness, a matt effect and uniformity to the makeup result. The fillers may be mineral or organic in any form, platelet-shaped, spherical or oblong. Non-limiting mention may be made of talc, mica, silica, kaolin, polyamide powders, poly-β-alanine powder and polyethylene powder, powders of tetrafluoroethylene polymers, lauroyllysine, starch, boron nitride, hollow polymer microspheres such as those of polyvinylidene chloride/acrylonitrile, or acrylic acid copolymers and silicone resin microbeads, elastomeric polyorganosiloxane particles, precipitated calcium carbonate, magnesium carbonate, magnesium hydrocarbonate, hydroxyapatite, hollow silica microspheres, glass or ceramic microcapsules, and metal soaps derived from organic carboxylic acids containing from 8 to 22 carbon atoms and for example from 12 to 18 carbon atoms, for example zinc, magnesium or lithium stearate, zinc laurate or magnesium myristate.

As disclosed herein, the composition 34 may also comprise a polymer such as a film-forming polymer. According to the present disclosure, the term “film-forming polymer” means a polymer capable, by itself or in the presence of an auxiliary film-forming agent, of forming a continuous film that adheres to a support, such as to keratin materials. Among the film-forming polymers that may be used in the composition 34 of the present disclosure, non-limiting mention may be made of synthetic polymers, of free-radical type or of polycondensate type, polymers of natural origin and mixtures thereof, for instance acrylic polymers, polyurethanes, polyesters, polyamides, polyureas and cellulose-based polymers, for instance nitrocellulose.

The composition 34 according to the present disclosure may also contain ingredients commonly used in cosmetics, such as vitamins, thickeners, gelling agents, trace elements, softeners, sequestering agents, fragrances, acidifying or basifying agents, preserving agents, sunscreens, surfactants, antioxidants, agents for preventing hair loss, antidandruff agents, propellants and ceramides and mixtures thereof.

The hair coloration composition 34 may further include a surface modification agent 40 that is attached to the surface 12 of each particle 10 and configured to bind each particle 10 to hair. For example, the surface modification agent 40 may be a protein such as a hydrolyzed keratin or partially hydrolyzed keratin, as is well known in the hair care field. The composition 34 may also include a hair modification agent 42 positioned in the medium and configured to enhance deposition of the particles 10 in the hair. The hair modification agent 42 may be any of the known enhancement agents used in the hair care field.

A person skilled in the art will take care to select this or these optional additional compounds, and/or the amount thereof, such that the beneficial properties of the composition according to the present disclosure are not, or are not substantially, adversely affected by the addition.

The composition 34 according to the present disclosure may be in the form of a suspension, a dispersion, for example oil-in-water by means of vesicles; an optionally thickened or even gelled aqueous or oily solution; an oil-in-water, water-in-oil or multiple emulsion; a gel or a mousse; an oily or emulsified gel; a dispersion of vesicles, such as, for example, lipid vesicles; a two-phase or multiphase lotion; a spray; a free, compact or cast powder; or an anhydrous paste. The composition as disclosed herein may have the appearance of a lotion, a cream, a salve, a soft paste, an ointment, a cast or molded solid, for example in stick or dish form, or a compacted solid.

In certain embodiments, particles 10 for the hair coloration composition 34 are formed from a block co-polymer material of the general formula (A-B)ₙ, where n is an integer. The polydispersity index (pdi) of each layer A and B is less than 1.05, preferably less than 1.02, and most preferably about 1.00. With a pdi of about 1.00, the block layers are nearly monodisperse, which assures that polymeric structures of precisely controlled periodicity can be obtained. Further, the refractive index difference between layers A and B is at least 0.3, and preferably greater than 0.5.

An example of a block co-polymer material forming the particles 10 is a polyelectrolyte gel that forms the first layer 14 and poly-(2-vinyl pyridine) swollen in water that forms the second layer 16.

In another example, the first layer material 14 is formed by polymer sodium polyacrylate gelled with sufficient water to yield a refractive index of 1.1, and the second layer material is polystyrene having a refractive index of 1.59. According to the equation

d_{B}=λ/2n_{B}

in order to display a photonic band gap centered at 400 nm (violet coloration), the second layer 16 is created with a thickness of 125 nm. In order to display a photonic band gap centered at 500 nm (green coloration), the second layer 16 is created with a thickness of 156 nm. In order to display a photonic band gap centered at 600 nm (orange coloration), the second layer 16 is created with a thickness of 188 nm. In order to display a photonic band gap centered at 700 nm (red coloration), the second layer 16 is created with a thickness of 219 nm. As may be seen, the thickness of the second layer 16 can be manipulated to create a photonic band gap at a selected portion of the visible spectrum.

Accordingly, a hair coloration composition for exhibiting a photonic band gap effect to provide selected coloration to hair has been provided. From the foregoing, it is to be appreciated that the exemplary embodiments of the hair coloration composition provide for the predetermination of light to be reflected to provide structural coloration based on wavelength. Further, the hair coloration compositions utilize coloration particles having sufficient largest dimensions to prevent absorption into the scalp, thereby reducing skin irritation.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the hair coloration compositions in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the appended claims and their legal equivalents.

1. A hair coloration composition comprising a plurality of particles, wherein each of the plurality of particles comprises:
a first layer of first material; and
a first layer of second material defining an interface with the first layer of first material, wherein the first material has a first refractive index and the second material has a second refractive index, wherein the difference between the first ref refractive index and the second refractive index is greater than zero, and wherein each of the plurality of particles is configured to exhibit a photonic band gap effect at the interface when exposed to incident white light,
wherein each of the plurality of particles has a largest dimension between about 10 microns and about 1000 microns.

2. The hair coloration composition of claim 1 wherein the difference between the first refractive index and the second refractive index is at least 0.05.

3. The hair coloration composition of claim 2 wherein the difference between the first refractive index and the second refractive index is at least 0.4.

4. The hair coloration composition of claim 3 wherein the difference between the first refractive index and the second refractive index is at least 0.5.

5. The hair coloration composition of claim 1 wherein the first refractive index is between 0.9 and 1.1.

6-7. (canceled)

8. The hair coloration composition of claim 7 wherein each of the plurality of particles has a largest dimension between about 25 microns and about 250 microns.

9. The hair coloration composition of claim 1 wherein the first layer of first material and the first layer of second material each have a thickness between about 1 nm and 1000 nm.

10. The hair coloration composition of claim 9 wherein the first layer of first material and the first layer of second material each have a thickness between about 2.5 nm and about 500 nm.

11. The hair coloration composition of claim 10 further comprising a second layer of first material and a second layer of second material.

12. The hair coloration composition of claim 1 wherein the first layer of first material and the first layer of second material are selected from the group consisting of polymers, inorganic materials, and self-organized organic materials.

13. The hair coloration composition of claim 1 wherein the first layer of first material and the first layer of second material form a block co-polymer having the formula (A−B)ₙ, wherein n is an integer.

14. The hair coloration composition of claim 13 wherein the first layer of first material is polystyrene and the first layer of second material is poly-(2-vinyl pyridine) swollen in water.

15. The hair coloration composition of claim 13 wherein the first layer of first material is polystyrene and wherein the first layer of second material is polymer sodium acrylate gelled with water.

16. A hair coloration composition comprising:
a plurality of particles, wherein each particle comprises alternating first and second layers respectively formed from a first material and a second material and defining interfaces therebetween, wherein the first material has a first refractive index and the second material has a second refractive index, wherein the difference between the first refractive index and the second refractive index is greater than zero, and wherein each is configured to exhibit a photonic band gap effect at the interfaces when, exposed to incident white light; and
a medium for holding the plurality of particles,
wherein each of the plurality of particles has a largest dimension between about 10 microns and about 1000 microns.

17. The hair coloration composition of claim 16 wherein each layer has a thickness, and wherein the first material, the second material, and the thickness of each layer is selected to create a photonic band gap at a selected portion of the visible spectrum.

18. The hair coloration composition of claim 17 wherein the difference between the first refractive index and the second refractive index is at least 0.5, wherein the first refractive index is between 0.9 and 1.1, wherein each particle has a diameter between about 25 microns and 250 microns, and wherein the thickness of each layer is between about 5 nm and 300 nm.

19. The hair coloration composition of claim 16 wherein the first material and the second material form a block co-polymer having the formula (A-B)ₙ, wherein n is an integer.

20. A hair coloration composition for application to keratinous fibers, the hair coloration composition comprising at least one particle in a medium, wherein the particle comprises alternating first and second layers respectively formed from a first material and a second material and defining interfaces therebetween, wherein the first material has a first refractive index and the second material has a second refractive index, wherein the difference between the first refractive index and the second refractive index is greater than at least about 0.5, wherein the particle is configured to exhibit a photonic band gap effect at the interfaces when exposed to incident white light to provide a predetermined structural coloration, wherein each of the plurality of particles has a largest dimension between about 10 microns and about 1000 microns.
